(19) United States
(12) Patent Application Publication
(10) Pub. No.: US 2025/0352101 A1
(43) Pub. Date: Nov. 20, 2025

(54) NEEDLE STICK INJURY PREVENTION DEVICE FOR BLOOD COLLECTION DEVICE TO PREVENT NEEDLE FROM BEING PUSHED OUT

(71) Applicant: Hsi-Chin Tsai, New Taipei City (TW)

(72) Inventors: Hsi-Chin Tsai, New Taipei City (TW); Ren-Hong Wang, Tainan City (TW)

(21) Appl. No.: 19/203,186

(22) Filed: May 9, 2025

(30) Foreign Application Priority Data
May 17, 2024 (TW) 113118457

(51) Int. Cl. A61B 5/15

(52) U.S. Cl. CPC A61B 5/150633; A61B 5/150343; A61B 5/150732

(57) ABSTRACT

A needle stick injury prevention device for blood collection device to prevent needle from being pushed out comprises a vacuum bottle support tube and a protective cover. The vacuum bottle support tube comprises a main body and a cover body, the cover body is arranged on one side of the opening of the main body and has a clamping portion with a groove and a protrusion, the groove having a positioning hole, the protrusion arranged on the bottom wall of the groove and having an arc surface. The bottom of the protective cover is arranged in the groove and has a first spring sheet, the outer side wall of the first spring sheet has a first barb, the first barb abuts against the arc surface to stretch the first spring sheet outward, and the top of the first barb abuts against the top wall of the positioning hole.

### CROSS-REFERENCE TO RELATED APPLICATION

This application claims the priority of Taiwanese patent application No. 113118457, filed on May 17, 2024, which is incorporated herewith by reference.

### BACKGROUND OF THE INVENTION

### 1. Field of the Invention

The present invention relates generally to a needle stick injury prevention structure, and more particularly, to a needle stick injury prevention device for a blood collection device to prevent a needle from being pushed out.

### 2. The Prior Arts

A blood collection device includes a plurality of bag bodies, a plurality of infusion tubes, two Y-type connectors, a steel needle, a blood filter, a protective cover, a needle cover, and a vacuum bottle support tube. The bag bodies include a main bag, a blood bag, a first sub-bag, a second sub-bag and a red blood cell preservation solution bag. The infusion tubes include a first infusion tube, a second infusion tube, a third infusion tube, a first connecting tube, a second connecting tube, and a third connecting tube. One of the Y-type connectors is connected to the first infusion tube, the second infusion tube, and the third infusion tube, and the other Y-type connector is connected to the first connecting tube, the second connecting tube, and the third connecting tube. The main bag is connected to the first infusion tube, the steel needle is connected to the second infusion tube, and the blood bag is connected to the third infusion tube and the vacuum bottle support tube. The protective cover is arranged on the second infusion tube, and the needle cover is arranged in the protective cover and receives the steel needle therein. The first connecting tube connects the main bag and the first sub-bag, and the blood filter is arranged on the first connecting tube. The second connecting tube is connected to the second sub-bag. The third connecting tube is connected to the red blood cell preservation solution bag.

The vacuum bottle support tube includes a main body and a cover body, the main body is cylindrical, and the cover body has an engaging portion. After completing the blood drawing, the steel needles that will become waste are stored in the protective cover, and the hook of the protective cover is engaged with the hook of the engaging portion of the cover body, so that each steel needle can be discarded together with the vacuum bottle support tube and the protective cover.

However, the hook of the engaging portion lacks elasticity, resulting in a poor effect of the hook in bearing external force. Therefore, as long as the user applies a slight external force to the engaging portion, the hook of the engaging portion cannot automatically adjust the interference area according to the external force, the hook of the protective cover and the hook of the engaging portion will automatically separate, and the steel needle will easily slip out of the protective cover and becomes exposed, increasing the risk of accidental puncture or blood infection.

Furthermore, the user cannot identify whether the needle seat is fixed inside the protective cover, and there is still a concern that the steel needle cannot be identified whether it is indeed retracted and fixed or exposed outside the protective cover.

### SUMMARY OF THE INVENTION

A primary objective of the present invention is to provide a needle stick injury prevention device for a blood collection device to prevent the needle from being pushed out, so as to ensure that the protective cover or the needle cover cannot be separated from the cover body.

Another objective of the present invention is to provide a needle stick injury prevention device for a blood collection device to prevent the needle from being pushed out, which can identify that the needle seat is indeed fixed inside the protective cover.

In order to achieve the aforementioned objectives, the present invention provides a needle stick injury prevention device for a blood collection device to prevent a needle from being pushed out, comprising: a vacuum bottle support tube, a protective cover, and a needle cover; wherein the vacuum bottle support tube further comprises a main body and a cover body, the cover body is arranged on one side of an opening of the main body and has a clamping portion, the clamping portion has a groove and a protrusion, the side wall of the groove is provided with a plurality of positioning holes, the protrusion is arranged on the bottom wall of the groove and is provided with a plurality of arc surfaces, and the arc surfaces are respectively aligned with the positioning holes; the bottom of the protective cover is arranged in the groove and is provided with a plurality of first spring sheets, and the outer wall of each first spring sheet has a first barb, and the first barbs abut against the arc surfaces so that the first spring sheets are stretched outward, and the top ends of the first barbs abut against the top walls of the positioning holes respectively; and the needle cover is arranged in the protection cover and is used for accommodating a steel needle therein.

In some embodiments, a connecting portion is formed between two adjacent positioning holes, and the connecting portions connect the side wall and the bottom wall of the groove; a plane is formed between two adjacent arc surfaces, the bottom of the protective cover is provided with a plurality of second spring sheets, the outer side wall of each second spring sheet has a second barb, and the second barbs are arranged on the outside of the planes.

In some embodiments, the needle stick injury prevention device for a blood collection device to prevent a needle from being pushed out further comprises a needle seat, the bottom of the needle seat is used for the steel needle to be set thereon, and the top of the needle seat is provided with a flange, and the flange on the top of the needle seat abuts against the top surface of the protective cover.

In some embodiments, the top of the protective cover has a conical portion, the conical portion comprises a plurality of shrapnel bodies, a groove is formed between two adjacent shrapnel bodies, and the flange at the top of the needle seat abuts against the top surface of the conical portion.

In some embodiments, a hook portion is protruding from the inner side wall of each shrapnel body, and a positioning groove is provided on the outer side wall of the needle seat; the positioning groove is located below the flange at the top of the needle seat, and the hook portion is located in the positioning groove.

In some embodiments, the hook portion comprises an extension portion and a positioning portion, the extension portion radially extends from the inner side wall of each of the shrapnel bodies, the positioning portion extends inward and downward or upward from one end of the extension portion, and the positioning portion is located in the positioning groove.

In some embodiments, the top of the protective cover has a conical portion and a cylindrical portion, the cylindrical portion is arranged on the top of the conical portion, and the flange at the top of the needle seat abuts against the top surface of the cylindrical portion.

The effect of the present invention is that when an external force pulls the protective cover, the first spring sheets are elastic and can automatically adjust the contact surface between the first barbs and the top wall of the positioning holes in response to the pulling or pushing of the external force. The contact surface of the first barbs will automatically adjust and shrink the gap between the first spring sheets due to the pulling or pushing force, so that the protective cover cannot be separated from the cover body due to the resistance of the contact surface between the first barbs and the top wall of the positioning holes, thereby preventing the needle cover and the steel needle from being pushed out outside the protective cover, so as to prevent medical staff or medical waste disposal personnel from being punctured and infected.

Furthermore, when an external force pushes the needle seat, the flange on the top of the needle seat is stuck by the conical portion, and the conical portion cannot be opened again, thereby preventing the needle cover and the steel needle from being pushed out outside the protective cover.

In addition, the user can recognize that the needle seat is indeed fixed inside the outer cover through the flange on the top of the needle seat located above the conical portion, and there is no need to worry that the needle cover and the steel needle will be pushed out or exposed outside the protective cover.

In order to achieve the aforementioned objectives, the present invention provides a needle stick injury prevention device for a blood collection device to prevent a needle from being pushed out, comprising a vacuum bottle support tube, a protective cover, and a needle cover; wherein the vacuum bottle support tube comprises a main body and a cover body, the cover body is arranged on one side of an opening of the main body and is provided with a clamping portion and a clamping slot, the clamping portion is arranged on the bottom wall of the clamping slot and is provided with a groove and a plurality of spring sheets, the spring sheets are arranged on the inner side wall of the groove, and each of the spring sheets extends inward and downward from the inner side wall of the groove; the bottom of the protective cover is arranged in the clamping slot; the needle cover is arranged in the protective cover to receive the steel needle therein, and is provided with a convex column at the bottom thereof, and the outer side wall of the convex column has a barb, and the top end of the barb abuts against the bottom ends of the spring sheets.

In some embodiments, the cover body comprises a clamping slot and a plurality of ribs, the ribs are arranged on the inner wall of the clamping slot, and the bottom of the protective cover is provided with a limiting portion, the outer side wall of the limiting portion contacts the ribs.

The effect of the present invention is that when an external force pulls the needle cover, the spring sheets are elastic and can automatically adjust the contact surface between the barbs and the spring sheets in response to the pulling or pushing of the external force. The contact surface of the spring sheets will automatically adjust and shrink the gap between the spring sheets due to the pulling or pushing force, and the resistance of the contact surface between the barbs and the spring sheets makes it impossible for the needle cover to separate from the cover body, thereby preventing the needle cover and the steel needle from being pushed out outside the protective cover, so as to prevent medical staff or medical waste disposal personnel from being punctured and infected.

### BRIEF DESCRIPTION OF THE DRAWINGS

The present invention will be apparent to those skilled in the art by reading the following detailed description of a preferred embodiment thereof, with reference to the attached drawings, in which:

FIG. 1 is a perspective view of a first embodiment of the present invention.

FIG. 2 is an exploded view of the first embodiment of the present invention.

FIG. 3 is a cross-sectional view of the first embodiment of the present invention.

FIG. 4A is a schematic view of region A of FIG. 3.

FIG. 4B is a schematic view of region B of FIG. 3.

FIG. 5A is a schematic view of blood drawing according to the first embodiment of the present invention.

FIG. 5B is a schematic view of the first embodiment of the present invention showing the needle seat being retracted and preventing needle stick injuries after use.

FIG. 6A is a perspective view of a protective cover according to a second embodiment of the present invention.

FIG. 6B is a cross-sectional view of a protective cover according to a second embodiment of the present invention.

FIG. 7A is a schematic view of region C of FIG. 6B.

FIG. 7B is a schematic view of FIG. 7A including a needle seat.

FIG. 8A is a perspective view of a protective cover according to a third embodiment of the present invention.

FIG. 8B is a cross-sectional view of a protective cover according to a third embodiment of the present invention.

FIG. 9A is a schematic view of region D of FIG. 8B.

FIG. 9B is a schematic view of FIG. 9A including a needle seat.

FIG. 10 is a perspective view of a fourth embodiment of the present invention.

FIG. 11 is an exploded view of a fourth embodiment of the present invention.

FIG. 12 is a cross-sectional view of a fourth embodiment of the present invention.

### DETAILED DESCRIPTION OF THE PREFERRED EMBODIMENT

The accompanying drawings are included to provide a further understanding of the invention, and are incorporated in and constitute a part of this specification. The drawings illustrate embodiments of the invention and, together with the description, serve to explain the principles of the invention.

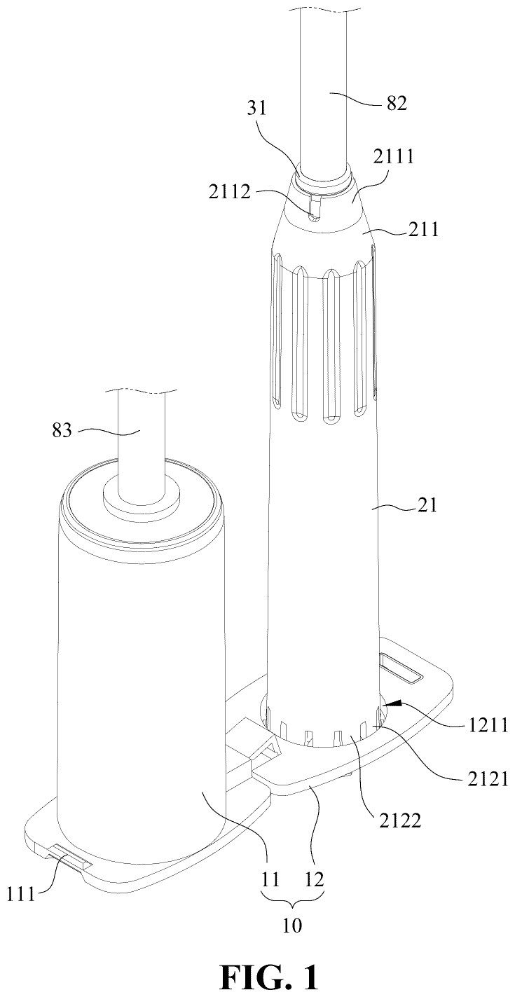

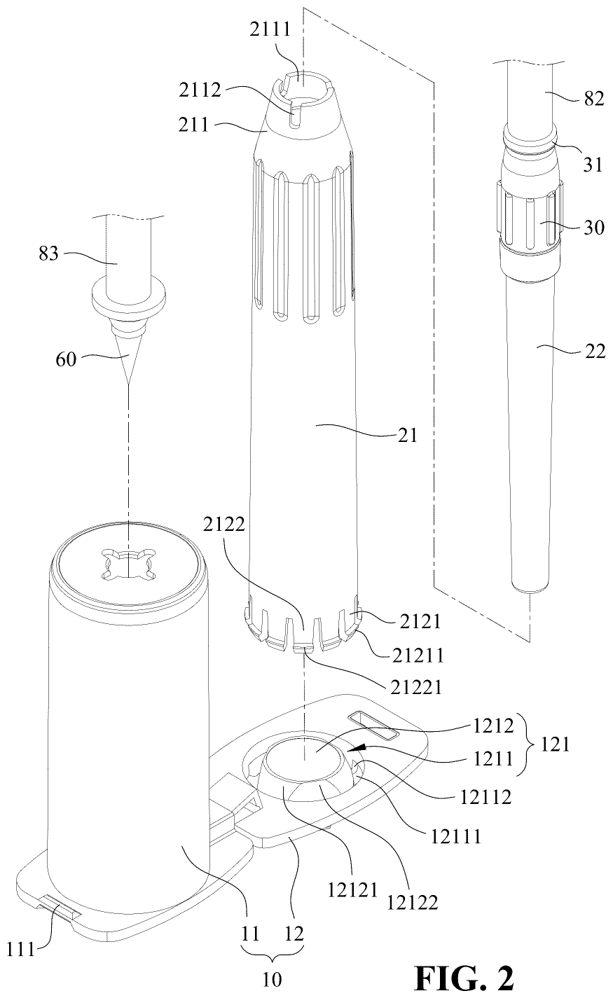

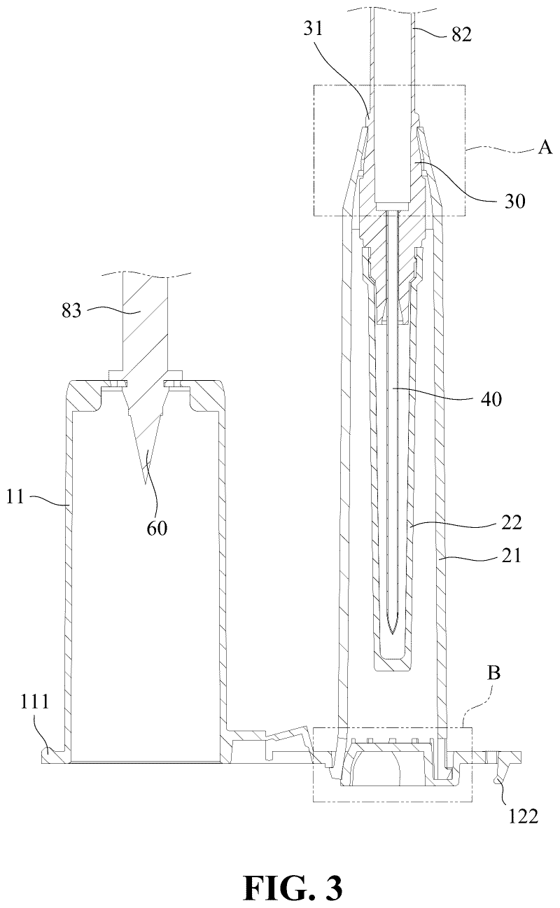

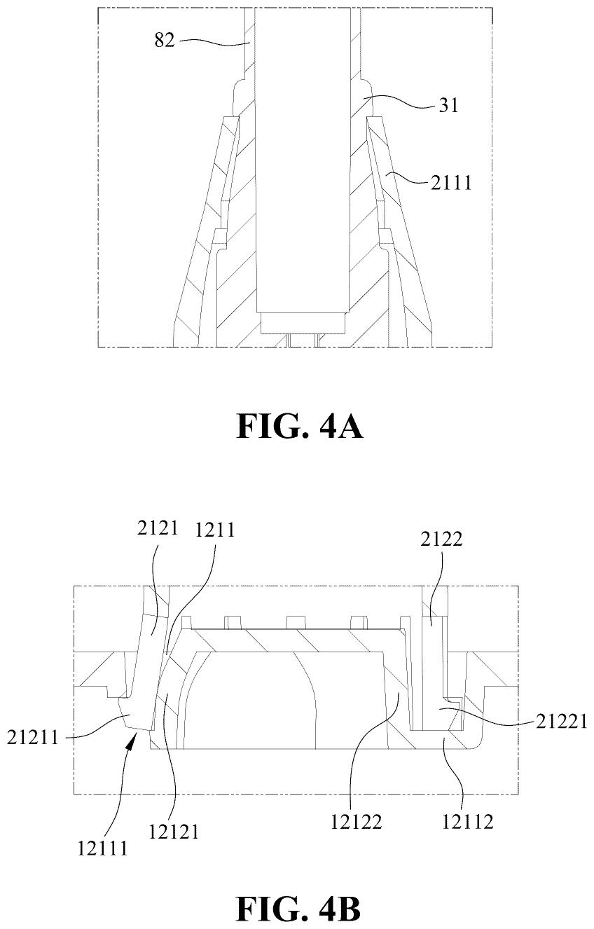

FIG. 1 is a perspective view of a first embodiment of the present invention, FIG. 2 is an exploded view of the first embodiment of the present invention, FIG. 3 is a cross-sectional view of the first embodiment of the present invention, FIG. 4A is a schematic view of region A of FIG. 3, and FIG. 4B is a schematic view of region B of FIG. 3. As shown in FIGS. 1 to 4B, the present invention provides a needle stick injury prevention device for a blood collection device to prevent a needle from being pushed out, comprising a vacuum bottle support tube 10, a protective cover 21, a needle cover 22, and a needle seat 30. The vacuum bottle support tube 10 includes a main body 11 and a cover body 12. The cover body 12 is arranged on one side of an opening of the main body 11. A buckle portion 111 is protruded from the other side of the opening of the main body 11. The cover body 12 has a clamping portion 121 and a clamping block 122. The clamping portion 121 has a groove 1211 and a protrusion 1212. A plurality of positioning holes 12111 are defined on the side wall of the groove 1211. A connecting portion 12112 is formed between two adjacent positioning holes 12111. The connecting portions 12112 connect the side wall and the bottom wall of the groove 1211. The protrusion 1212 is disposed on the bottom wall of the groove 1211 and is provided with a plurality of arc surfaces 12121. The arc surfaces 12121 are respectively aligned with the positioning holes 12111, and a plane 12122 is formed between two adjacent arc surfaces 12121. The top of the protective cover 21 has a conical portion 211, which is a cone and includes a plurality of shrapnel bodies 2111. A groove 2112 is formed between two adjacent shrapnel bodies 2111. The bottom of the protective cover 21 is arranged in the groove 1211 and is provided with a plurality of first spring sheets 2121 and a plurality of second spring sheets. The outer wall of each first spring sheet 2121 has a first barb 21211, and the outer wall of each second spring sheet 2122 has a second barb 21221. The first barbs 21211 are pushed outward by the arc surfaces 12121 and their top ends abut against the top walls of the positioning holes 12111. The second barbs 21221 are arranged on the outside of the planes 12122. The needle cover 22 is disposed in the protection cover 21 and is used to receive a steel needle 40 therein. The bottom of the needle seat 30 is used for the steel needle 40 to be disposed thereon. The top of the needle seat 30 has a flange 31. The flange 31 on the top of the needle seat 30 abuts against the top surface of the conical portion 211.

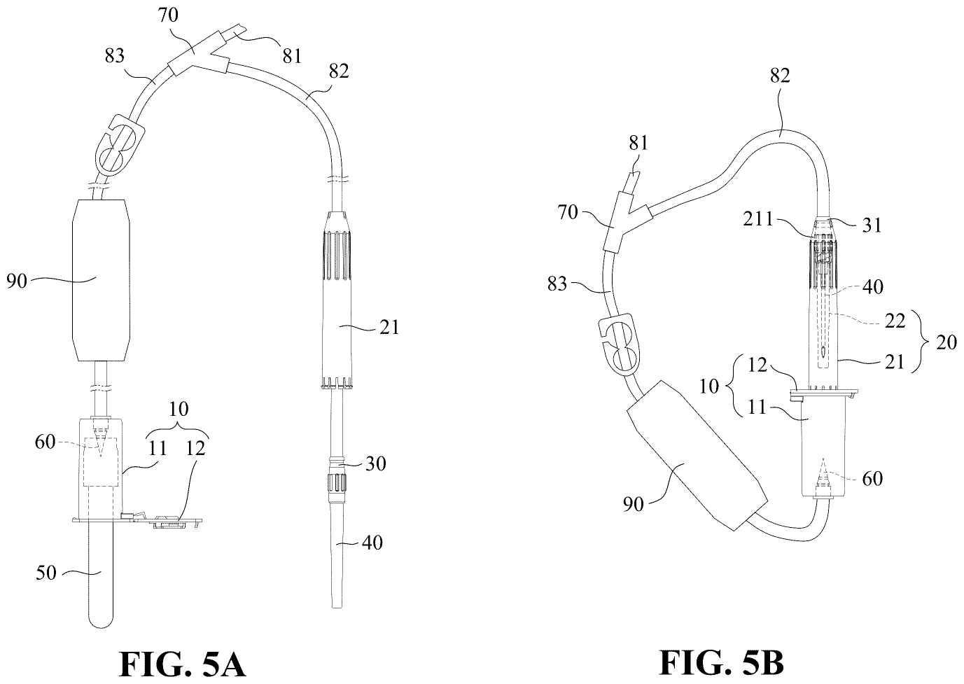

FIG. 5A is a schematic view of blood drawing according to the first embodiment of the present invention. As shown in FIG. 5A, the vacuum bottle support tube 10 and the protective cover 21 are in a separated state. The interior of the main body 11 is used to accommodate a vacuum bottle 50, and the top of the main body 11 is used for a puncture needle 60 to penetrate. The puncture needle 60 pierces the bottle cap of the vacuum bottle 50 and enters the vacuum bottle. A Y-type connector 70 connects a first infusion tube 81, a second infusion tube 82, and a third infusion tube 83, a main bag (not shown) is connected to the first infusion tube 81, the needle seat 30 is connected to the second infusion tube 82, and a blood bag 90 is connected to the main body 11 and the third infusion tube 83. A first connecting tube (not shown) connects the main bag and a first sub-bag (not shown), a blood filter (not shown) is arranged on the first connecting tube, a second connecting tube (not shown) is connected to a second sub-bag (not shown), and a third connecting tube (not shown) is connected to a red blood cell preservation solution bag (not shown). The needle seat 30 is pushed so that the needle seat 30, the needle cover 22, and the steel needle 40 are located outside the protective cover 21, and then the needle cover 22 is removed so that the steel needle 40 is exposed outside the protective cover 21, and blood drawing can be performed at this time.

FIG. 5B is a schematic view of the first embodiment of the present invention showing the needle seat being retracted and preventing needle stick injuries after use. As shown in FIG. 5B, and also referring to FIG. 3 and FIG. 4B, after completing the blood drawing, first, the needle cover 22 is set on the needle seat 30 so that the needle cover 22 covers the steel needle 40; then, the needle seat 30 is moved upward, and the flange 31 on the top of the needle seat 30 props up the shrapnel bodies 2111 on the top of the protective cover 21 until the flange 31 on the top of the needle seat 30 abuts against the top surface of the conical portion 211. At this time, the needle cover 22 and the steel needle 40 are located inside the protective cover 21; then, remove the vacuum bottle 50, and close the opening of the main body 11 with the cover body 12, and the clamping block 122 is embedded in the buckle portion 111; finally, set the bottom of the protective cover 21 in the groove 1211, the first barbs 21211 are pushed outward by the arc surfaces 12121 and the top ends thereof are against the top walls of the positioning holes 12111, and the second barbs 21221 are arranged on the outside of the planes 12122.

Thereby, when an external force pulls or pushes the protective cover 21, the first spring sheets 2121 are elastic and can automatically adjust the contact surface between the first barbs 21211 and the top wall of the positioning holes 12111 in response to the pulling or pushing of the external force. The contact surface of the first barbs 21211 will automatically adjust and shrink the gap between the first spring sheets 2121 due to the pulling or pushing force, and the protection cover 21 will not be separated from the cover body 12 due to the resistance of the contact surface between the first barbs 21211 and the top wall of the positioning holes 12111, thereby preventing the needle cover 22 and the steel needle 40 from being pushed out outside the protection cover 21, so as to prevent medical staff or medical waste disposal personnel from being punctured and infected.

Furthermore, when an external force pushes the needle seat 30, the flange 31 on the top of the needle seat 30 is stuck by the conical portion 211 and cannot open the conical portion 211 again, thereby preventing the needle cover 22 and the steel needle 40 from being pushed out outside the protective cover 21.

In addition, the user can recognize that the needle seat 30 is indeed fixed inside the protective cover 21 through the flange 31 on the top of the needle seat 30 located above the conical portion 211, and there is no need to worry that the needle cover 22 and the steel needle 40 will be pushed out or exposed outside the protective cover 21.

In addition, the provision of the second spring sheets 2122 can make the mold of the protection cover 21 easier to design and the protection cover 21 easier to manufacture.

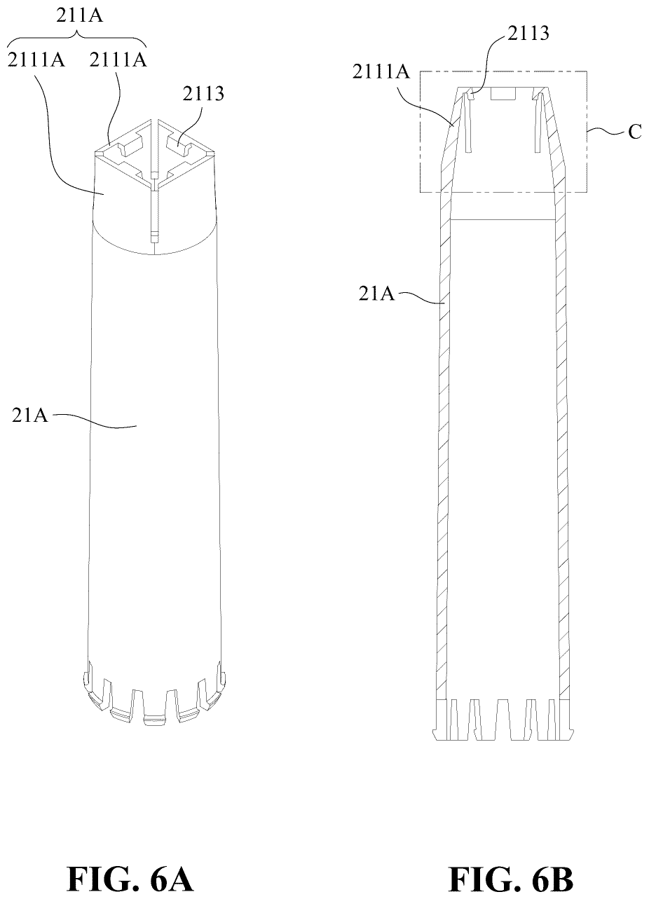

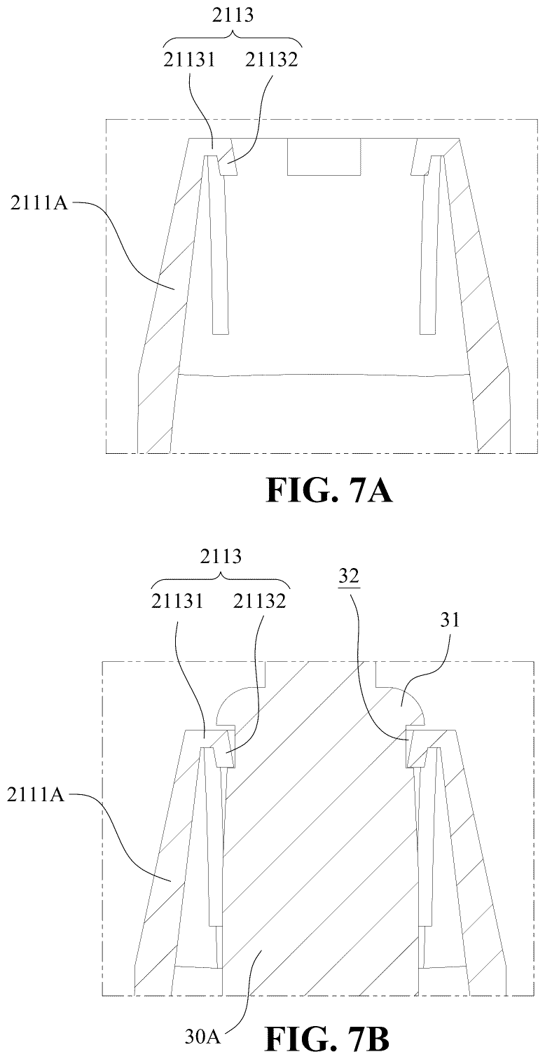

FIG. 6A is a perspective view of a protective cover according to a second embodiment of the present invention, FIG. 6B is a cross-sectional view of a protective cover according to a second embodiment of the present invention, FIG. 7A is a schematic view of region C of FIG. 6B, and FIG. 7B is a schematic view of FIG. 7A including a needle seat. As shown in FIGS. 6A, 6B, 7A, and 7B, the difference between the second embodiment and the first embodiment is that: first, the conical portion 211A is in the shape of a pyramid; second, a hook portion 2113 is convexly provided on the inner wall of each shrapnel body 2111A, and a positioning groove 32 is provided on the outer wall of the needle seat 30A. The positioning groove 32 is located below the flange 31 at the top of the needle seat 30A, and the hook portion 2113 is located in the positioning groove 32; third, the hook portion 2113 comprises an extension portion 21131 and a positioning portion 21132, the extension portion 21131 radially extends from the inner wall of each shrapnel body 2111A, the positioning portion 21132 extends inward and downward from one end of the extension portion 21131, and the positioning portion 21132 is located in the positioning groove 32. Thereby, when an external force pulls the needle seat 30, the needle seat 30A is stuck by the positioning portion 21132, so that the needle seat 30A will not be separated from the protective cover 21A, thereby preventing the needle cover 22 and the steel needle 40 from being pushed out outside the protective cover 21A.

In some embodiments, the positioning portion 21132 may also extend inward and upward from one end of the extension portion 21131, and achieves the same effect as that of the positioning portion of the second embodiment.

In some embodiments, the hook portion 2113 is in the shape of a barb, and achieves the same effect as that of the second embodiment.

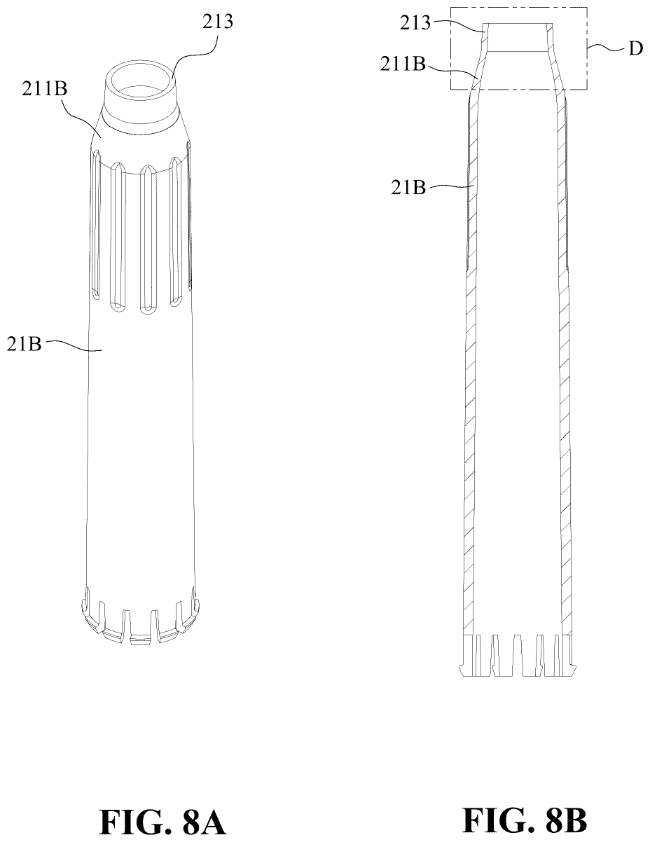

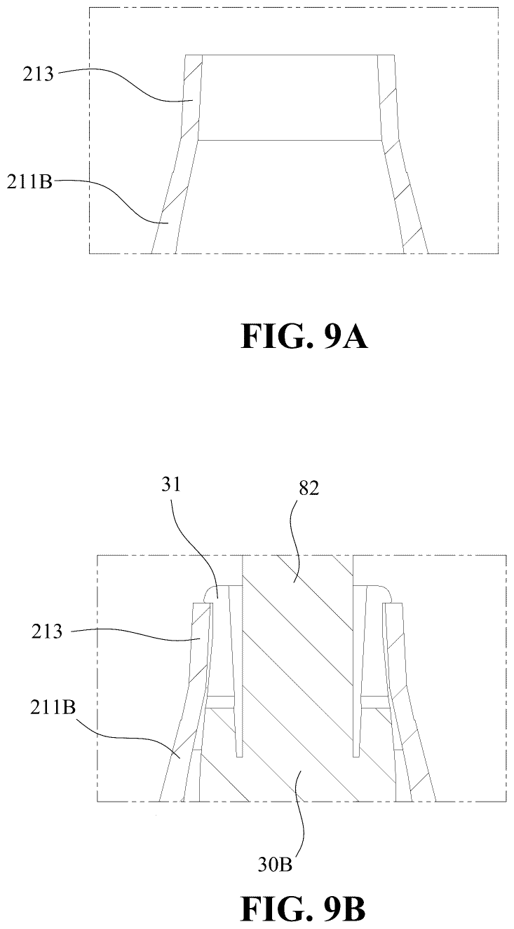

FIG. 8A is a perspective view of a protective cover according to a third embodiment of the present invention, FIG. 8B is a cross-sectional view of a protective cover according to a third embodiment of the present invention, FIG. 9A is a schematic view of region D of FIG. 8B, and FIG. 9B is a schematic view of FIG. 9A including a needle seat 30B. As shown in FIGS. 8A, 8B, 9A, and 9B, the differences between the third embodiment and the first embodiment are: first, the conical portion 211B does not have a shrapnel body and a groove; second, the top of the protective cover 21B further includes a cylindrical portion 213, the cylindrical portion 213 is arranged on the top of the conical portion 211B, and the flange 31 abuts against the top surface of the cylindrical portion 213. The third embodiment achieves the same effects as those of the first embodiment.

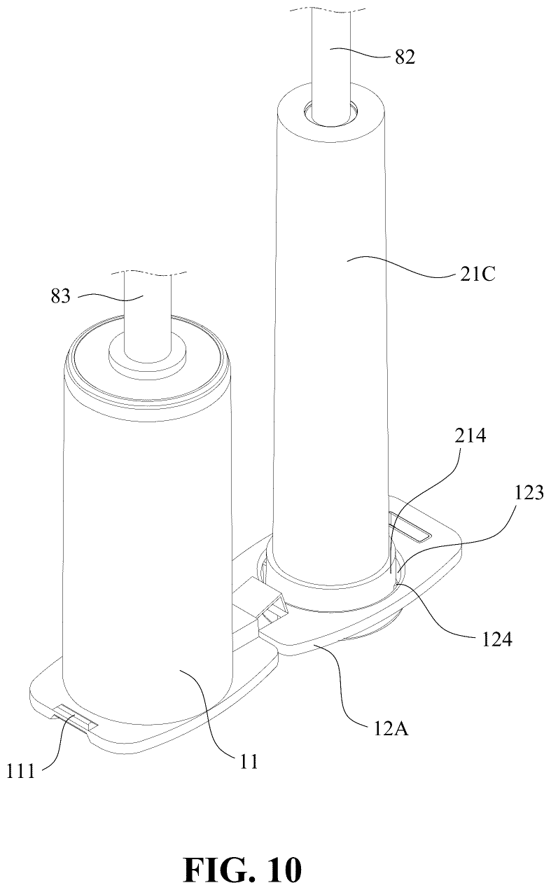

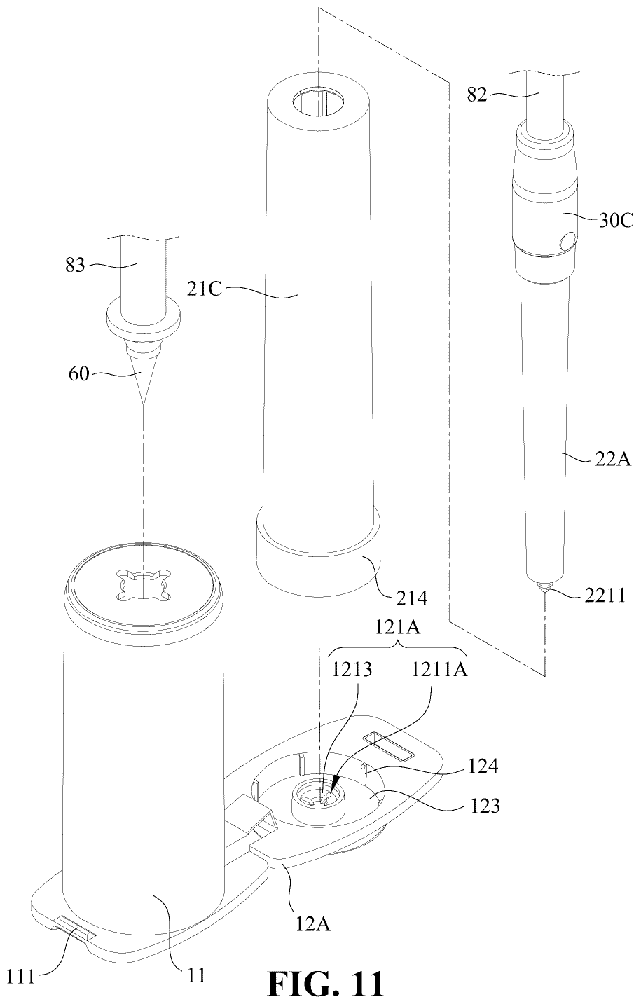

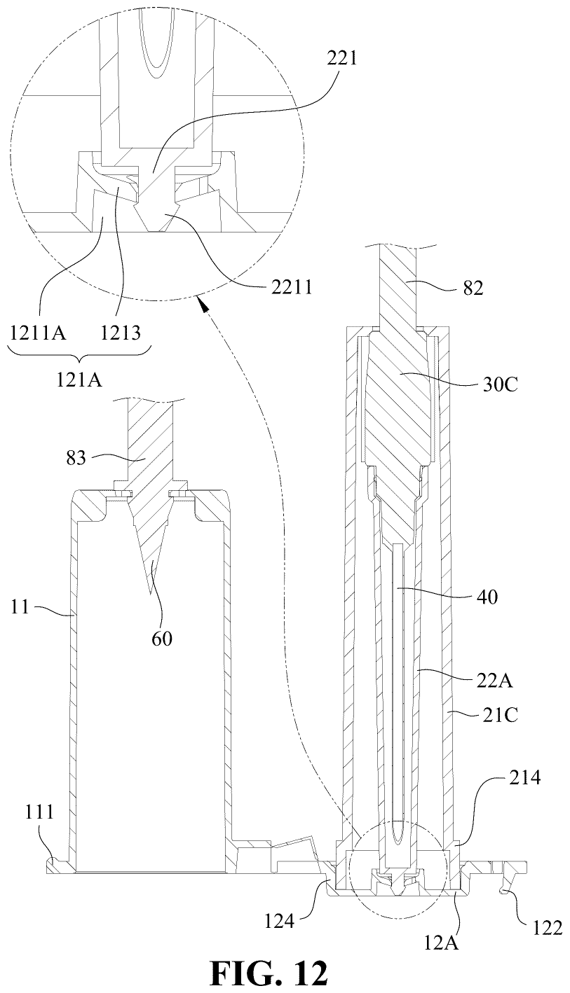

FIG. 10 is a perspective view of a fourth embodiment of the present invention, FIG. 11 is an exploded view of a fourth embodiment of the present invention, and FIG. 12 is a cross-sectional view of a fourth embodiment of the present invention. As shown in FIGS. 10, 11, and 12, the difference between the fourth embodiment and the first embodiment is that: first, the cover body 12A has a clamping slot 123 and a plurality of ribs 124, and the ribs 124 are disposed on the inner side wall of the clamping slot 123; second, the bottom of the protective cover 21C is disposed in the clamping slot 123, and the bottom of the protective cover 21C has a limiting portion 214, and the outer side wall of the limiting portion 214 contacts the ribs 124; third, the engaging portion 121A is disposed on the bottom wall of the clamping slot 123 and has a plurality of spring sheets 1213, and each spring sheet 1213 extends inwardly and downwardly from the inner side wall of the groove 1211A; fourth, a convex column 221 is convexly provided on the bottom of the needle cover 22A, and the outer side wall of the convex column 221 has a barb 2211; fifth, the needle seat 30C does not have a flange.

After completing blood drawing, first, set the needle cover 22A on the needle seat 30C so that the needle cover 22A covers the steel needle 40; then, move the needle seat 30C upward until the needle cover 22A and the steel needle 40 are located inside the protective cover 21C; then, remove the vacuum bottle 50, and close the opening of the main body 11 with the cover body 12A, and the clamping block 122 is embedded in the buckle portion 111; finally, set the bottom of the protective cover 21C in the clamping slot, the outer side wall of the limiting portion 214 contacts the ribs 124, and the top of the barb 2211 abuts against the bottom end of the spring sheets 1213.

Thus, when an external force pulls or pushes the needle cover 22A, the spring pieces 1213 are elastic and can automatically adjust the contact surface between the barbs 2211 and the spring sheets 1213 in response to the pulling or pushing of the external force. The contact surface of the spring sheets 1213 will automatically adjust and shrink the gap between the spring pieces 1213 due to the pulling or pushing force. The resistance of the contact surface between the barbs 2211 and the spring sheets 1213 makes it impossible for the needle cover 22A to separate from the cover body 12A, thereby preventing the needle cover 22A and the steel needle 40 from being pushed out outside the protective cover 21C, so as to prevent medical staff or medical waste disposal personnel from being punctured and infected.

Furthermore, the ribs 124 can enhance the effect of fixing the protective cover 21C to the clamping slot 123, thereby preventing the protective cover 21C from being separated from the cover body 12A.

Although the present invention has been described with reference to the preferred embodiments thereof, it is apparent to those skilled in the art that a variety of modifications and changes may be made without departing from the scope of the present invention which is intended to be defined by the appended claims.

What is claimed is:

1. A needle stick injury prevention device for blood collection device to prevent needle from being pushed out, comprising:
a vacuum bottle support tube, further comprising: a main body and a cover body, the cover body being arranged on one side of an opening of the main body and having a clamping portion, the clamping portion having a groove and a protrusion, the side wall of the groove being provided with a plurality of positioning holes, the protrusion being arranged on the bottom wall of the groove and provided with a plurality of arc surfaces, and the arc surfaces being respectively aligned with the positioning holes;
a protective cover, with a bottom arranged in the groove and provided with a plurality of first spring sheets, and the outer wall of each first spring sheet having a first barb, and the first barbs abutting against the arc surfaces so that the first spring sheets being stretched outward, and the top ends of the first barbs abutting against the top walls of the positioning holes respectively; and
a needle cover, arranged in the protection cover and being used for accommodating a steel needle therein.

2. The needle stick injury prevention device for blood collection device to prevent needle from being pushed out according to claim 1, wherein a connecting portion is formed between two adjacent positioning holes, and the connecting portions connect the side wall and the bottom wall of the groove; a plane is formed between two adjacent arc surfaces, the bottom of the protective cover is provided with a plurality of second spring sheets, the outer side wall of each second spring sheet has a second barb, and the second barbs are arranged on the outside of the planes.

3. The needle stick injury prevention device for blood collection device to prevent needle from being pushed out according to claim 1, further comprising a needle seat, the bottom of the needle seat being used for the steel needle to be set thereon, and the top of the needle seat being provided with a flange, and the flange on the top of the needle seat abutting against the top surface of the protective cover.

4. The needle stick injury prevention device for blood collection device to prevent needle from being pushed out according to claim 3, wherein the top of the protective cover has a conical portion, the conical portion comprises a plurality of shrapnel bodies, a groove is formed between two adjacent shrapnel bodies, and the flange at the top of the needle seat abuts against the top surface of the conical portion.

5. The needle stick injury prevention device for blood collection device to prevent needle from being pushed out according to claim 4, wherein a hook portion is protruding from the inner side wall of each shrapnel body, and a positioning groove is provided on the outer side wall of the needle seat; the positioning groove is located below the flange at the top of the needle seat, and the hook portion is located in the positioning groove.

6. The needle stick injury prevention device for blood collection device to prevent needle from being pushed out according to claim 5, wherein the hook portion comprises an extension portion and a positioning portion, the extension portion radially extends from the inner side wall of each of the shrapnel bodies, the positioning portion extends inward and downward or upward from one end of the extension portion, and the positioning portion is located in the positioning groove.

7. The needle stick injury prevention device for blood collection device to prevent needle from being pushed out according to claim 3, wherein the top of the protective cover has a conical portion and a cylindrical portion, the cylindrical portion is arranged on the top of the conical portion, and the flange at the top of the needle seat abuts against the top surface of the cylindrical portion.

8. A needle stick injury prevention device for blood collection device to prevent needle from being pushed out, comprising:
a vacuum bottle support tube, further comprising: a main body and a cover body, the cover body being arranged on one side of an opening of the main body and provided with a clamping portion and a clamping slot, the clamping portion being arranged on the bottom wall of the clamping slot and provided with a groove and a plurality of spring sheets, the spring sheets being arranged on the inner side wall of the groove, and each of the spring sheets extending inward and downward from the inner side wall of the groove;
a protective cover, with a bottom arranged in the clamping slot; and
a needle cover, arranged in the protective cover to receive the steel needle therein, and provided with a convex column at the bottom thereof, and the outer side wall of the convex column having a barb, and the top end of the barb abutting against the bottom ends of the spring sheets.

9. The needle stick injury prevention device for blood collection device to prevent needle from being pushed out according to claim 8, wherein the cover body comprises a clamping slot and a plurality of ribs, the ribs are arranged on the inner wall of the clamping slot, and the bottom of the protective cover is provided with a limiting portion, the outer side wall of the limiting portion contacts the ribs.
